(19) United States
(12) Patent Application Publication
(10) Pub. No.: US 2025/0275865 A1
(43) Pub. Date: Sep. 4, 2025

(54) AN UROSTOMY BAG WITH A SENSOR TO DETERMINE AND COMMUNICATE A URINE LEVEL TO USER

(71) Applicants: Sunil Kumar SINGH, Gurgaon (IN); Anil MANDHANI, Gurgaon (IN)

(72) Inventors: Sunil Kumar SINGH, Gurgaon (IN); Anil MANDHANI, Gurgaon (IN)

(21) Appl. No.: 18/709,525

(22) PCT Filed: Nov. 27, 2022
(86) PCT No.: PCT/IN2022/051034

§ 371 Date: May 13, 2024

(51) Int. Cl. A61F 5/445; A61F 5/44

(52) U.S. Cl. CPC A61F 5/445; A61F 5/4405

(57) ABSTRACT

Disclosed is a urostomy bag (100) for a user. The urostomy bag (100) includes a first chamber (102) disposed on a stoma of the user such that the first chamber (102) receives the urine of the user; a tube (104) having a first end (104A) and a second end (104B) such that the first end (104A) is received within the first chamber (102) to allow passage of the urine through the tube (104); a second chamber (110) having an inlet (110A) and coupled to the tube (104) such that the second end (104B) is received within the inlet (110A) to allow passage of the urine from the tube (104) to the second chamber (); and an electronic sensor unit (106) configured to alert the user when volume of the urine in the second chamber (110) reaches to a pre-determined volume of the second chamber (110). FIG. 1 is the reference figure

### TECHNICAL FIELD

The present disclosure relates generally to medical devices. More particularly, the present disclosure relates to urostomy bag with a sensor to determine and communicate urine level to a user.

### BACKGROUND

The urinary bladder stores urine thereby allowing urination to be infrequent and controlled. When the urinary bladder is removed or when the bladder function is affected adversely, there is a need to perform surgery in order to create an alternate passage for the urine. This urinary diversion in patients is created by making an outlet on the abdominal wall using small intestine or ureters brought in directly, in the form of urinary stoma. The stoma is a pathway from the kidneys to the outside of patient abdomen. The stoma is then connected to a urine collecting pouch, which requires timely disposal when it is filled.

Generally, the urine collection pouch is kept at the level of waist and it hangs freely from the waist. Once it is filled with urine, the patient empties the pouch by opening the outlet. This conventional pouch is not patient friendly, which may cause cringe-worthy moments for the patient either socially or personally. The pouch being a hanging bag that impacts dignified living of an individual as the hanging bags are sign of their health issues. Accumulation of urine in the bag causes bulging of the pouch and gives discomfort to the patient carrying a pouch filled with urine. Patients need to continuously monitor the bag or pouch for emptying and it's not a one hand operation. Emptying cycles are more frequent as pouch can hold up to 150 to 200 cc of urine. There is a need for a device that does not restrict the patient's movements and further enhances the comfort of the user. With the evolution of medical sciences, medical trousers have been used for urine incontinence that includes trousers and a urine storage bag. The urine mask and the urine storage bags are connected by a catheter. The urine mask is further provided with an elastic band that is snuggly fixed to the human body. The trousers provide space for the urine storage bag and social acceptance and decency. However, the users need to wear the trouser all the time, which creates discomfort to them. There is a need for a device that aids patients with a stoma to discharge urine in a way that looks dignified and close to the act of passing urine naturally.

Moreover, the current urine or urostomy bags need to be checked manually to determine level of urine at different times and since the urine bags are disposed inside the trousers or lowers, it is not always easy for a user to determine whether the bag is full or not

Therefore, there exists a need for a urine collecting device or a urostomy bag that solves the aforementioned problems of the conventional urostomy bags.

### SUMMARY

In view of the foregoing, a urostomy bag for a user is disclosed. The urostomy bag includes a first chamber disposed on a stoma of the user such that the first chamber receives the urine of the user; a tube having a first end and a second end such that the first end is received within the first chamber to allow passage of the urine through the tube; a second chamber having an inlet and an outlet and coupled to the tube such that the second end is received within the inlet to allow passage of the urine from the tube to the second chamber; and an electronic sensor unit disposed between the first end and the second end of the tube and configured to alert the user when volume of the urine in the second chamber reaches to a pre-determined volume of the second chamber.

In some embodiments, the electronic sensor unit includes one or more sensors configured to sense flow rate of the urine that flows through the tube to measure quantity of the urine collected inside the second chamber; a microcontroller coupled to the one or more sensors and configured to, based upon the quantity of the urine collected inside the second chamber, generates an alert signal to alert the user; and a battery that is coupled to the one or more sensors and the microcontroller and configured to supply electrical power to the one or more sensors and to the microcontroller.

In some embodiments, the one or more sensors comprises one of a flow rate sensor, a capacitive sensor, and a particle sensor.

In some embodiments, the microcontroller generates the alert signal when volume of the urine collected in the second chamber reaches to the pre-defined volume of the second chamber.

In some embodiments, the second chamber comprising a plurality of baffles arranged within the second chamber such that the plurality of baffles defines a cell inside the second chamber.

In some embodiments, the urostomy bag further includes a non-return valve that is disposed within the second chamber and coupled to the second end of the tube and adapted to prevent back-flow of the urine that passes through the tube.

In some embodiments, the urostomy bag further includes an outlet valve coupled to the outlet of the second chamber; and a push button coupled to the outlet valve, such that upon actuation of the push button, the outlet valve exhibits one of an open position and a closed position, wherein the closed position prevents drainage of the urine collected in the second chamber and the open position allows drainage of the urine collected in the second chamber.

In some embodiments, a system to alert a user is disclosed. The system includes a urostomy bag that includes a first chamber disposed on a stoma of the user such that the first chamber receives the urine of the user; a tube having a first end and a second end such that the first end is received within the first chamber to allow passage of the urine through the tube; a second chamber having an inlet and an outlet and coupled to the tube such that the second end is received within the inlet to allow passage of the urine from the tube to the second chamber; and an electronic sensor unit disposed between the first end and the second end of the tube and configured to alert the user when volume of the urine in the second chamber reaches to a pre-determined volume of the second chamber.

In some embodiments, the electronic sensor unit includes one or more sensors configured to sense flow rate of the urine that flows through the tube to measure quantity of the urine collected inside the second chamber; microcontroller coupled to the one or more sensors and configured to, based upon the quantity of the urine collected inside the second chamber, generates an alert signal to alert the user; and a battery that is coupled to the one or more sensors and the microcontroller and configured to supply electrical power to the one or more sensors and to the microcontroller.

In some embodiments, the system further includes a user device coupled to the urostomy bag, wherein the microcontroller generates the alert signal on the user device to alert the user.

### BRIEF DESCRIPTION OF DRAWINGS

The above and still further features and advantages of aspects of the present disclosure becomes apparent upon consideration of the following detailed description of aspects thereof, especially when taken in conjunction with the accompanying drawings, and wherein:

FIG. 1 illustrates a urostomy bag for a user, in accordance with an embodiment herein;

FIG. 2 illustrates a block diagram of an electronic sensor unit of the urostomy bag of FIG. 1, in accordance with an embodiment herein; and

FIG. 3 illustrates a block-diagram of a system to alert a user, in accordance with an embodiment herein.

To facilitate understanding, like reference numerals have been used, where possible, to designate like elements common to the figures.

### DETAILED DESCRIPTION

Various aspects of the present disclosure provide a urostomy bag for a user and a system to alert the user. The following description provides specific details of certain aspects of the disclosure illustrated in the drawings to provide a thorough understanding of those aspects. It should be recognized, however, that the present disclosure can be reflected in additional aspects and the disclosure may be practiced without some of the details in the following description.

The various aspects including the example aspects are now described more fully with reference to the accompanying drawings, in which the various aspects of the disclosure are shown. The disclosure may, however, be embodied in different forms and should not be construed as limited to the aspects set forth herein. Rather, these aspects are provided so that this disclosure is thorough and complete, and fully conveys the scope of the disclosure to those skilled in the art. In the drawings, the sizes of components may be exaggerated for clarity.

It is understood that when an element or layer is referred to as being “on,” “connected to,” or “coupled to” another element or layer, it can be directly on, connected to, or coupled to the other element or layer or intervening elements or layers that may be present. As used herein, the term “and/or” includes any and all combinations of one or more of the associated listed items.

The subject matter of example aspects, as disclosed herein, is described with specificity to meet statutory requirements. However, the description itself is not intended to limit the scope of this disclosure. Rather, the inventor/inventors have contemplated that the claimed subject matter might also be embodied in other ways, to include different features or combinations of features similar to the ones described in this document, in conjunction with other technologies. Generally, the various aspects including the example aspects relate to a urostomy bag for a user and a system to alert the user. As mentioned, there remains a need for an efficient urostomy bag and a system to alert the user about the urine level in the urostomy bag. The urostomy bag of the present disclosure provides an alert when a second chamber of the urostomy bag gets filled up to a pre-determined volume.

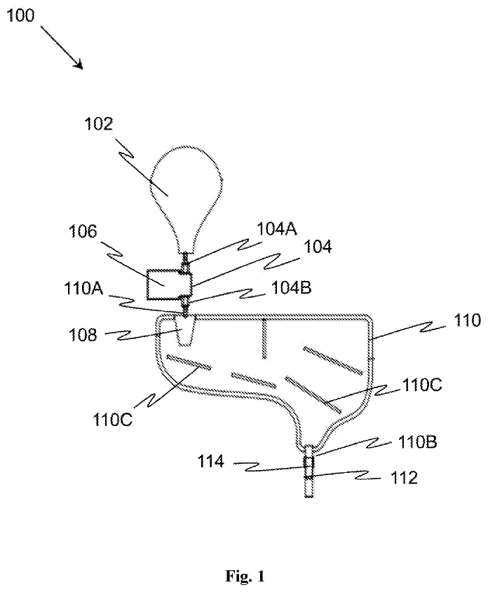

FIG. 1 illustrates a urostomy bag 100 for a user, in accordance with an embodiment herein. The urostomy bag 100 may include a first chamber 102, a tube 104, an electronic sensor unit 106, a non-return flow valve 108, a second chamber 110, an outlet valve 112, and a push button 114. The tube 104 may include a first end 104A and a second end 104B. The second chamber 110 may include an inlet 110A, an outlet 110B, and a plurality of baffles 110C.

In some embodiments, the tube 104 may be made up of silicone.

The electronic sensor unit 106 may be disposed between the first end 104A and the second end 104B of the tube 104. The non-return flow valve 108 may be disposed within the second chamber 110 and coupled to the second end 104B of the tube 104. The plurality of baffles 110C may be arranged within the second chamber 110 such that the plurality of baffles 110C define a cell inside the second chamber 110. The outlet valve 112 may be coupled to the outlet 110B of the second chamber 110. The push button 114 may be coupled to the outlet valve 112.

The first chamber 102 may be disposed on a stoma of the user such that the first chamber 102 receives the urine that is released from the stoma of the user. The first end 104A may be received within the first chamber 102 to allow passage of the urine from the first chamber 102 to the second chamber 110 through the tube 104. The second chamber 110 may be coupled to the tube 104 such that the second end 104B of the tube 104 is received within the inlet 110A of the second chamber 110 to allow passage of the urine from the tube 104 to the second chamber 110. Upon passage of the urine from the tube 104 to the second chamber 110, the second chamber 110 may gradually get filled with the urine. While passing the urine from the tube 104 to the second chamber 110, the non-return flow valve 108 may be adapted to prevent back-flow of the urine. The electronic sensor unit 106 may be configured to alert the user when volume of the urine in the second chamber 110 reaches to a pre-determined volume of the second chamber 110.

In some embodiments, value of the pre-determined volume of the second chamber 110 may be in a range between 300 milli-litre and 400 milli-litre. Preferably, the value of the pre-determined volume of the second chamber 110 may be 350 milli-litre.

In some embodiments, the plurality of baffles 110C may be adapted to control the rate of flow of the urine within the second chamber 110. The plurality of baffles 110C may therefore guide the urine within the second chamber 110 and hence preventing the turbulence that is caused by the accumulated urine within the second chamber 110.

The push button 114 may be adapted to actuate such that upon actuation of the push button 114, the outlet valve 112 may exhibit one of an open position and a closed position. The closed position of the outlet valve 112 may prevent drainage of the urine collected in the second chamber 110. The open position of the outlet valve 112 may allow drainage of the urine collected in the second chamber 110.

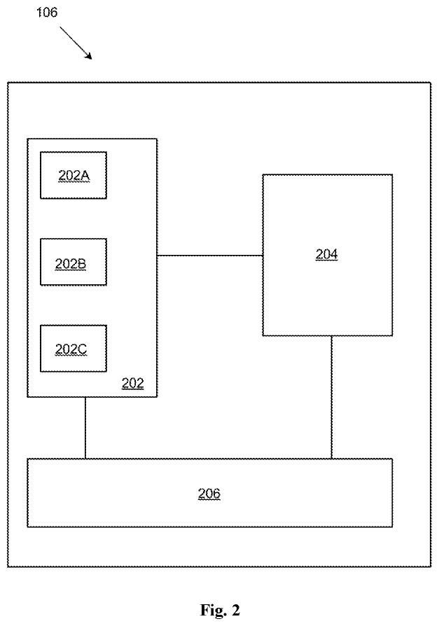

FIG. 2 illustrates a block diagram of an electronic sensor unit 106 of the urostomy bag 100 of FIG. 1, in accordance with an embodiment herein. The electronic sensor unit 106 may include one or more sensors 202 of which first through third sensors 202A-202C are shown, a micro-controller 204, and a battery 206. The one or more sensors 202 may be coupled to the micro-controller 204. The battery 206 may be coupled to the one or more sensors 202 and the micro-controller 204.

The one or more sensors 202 may be configured to sense flow rate of the urine that flows through the tube 104 to measure quantity of the urine collected inside the second chamber 110. The micro-controller 204 may be configured to, based upon the quantity of the urine collected inside the second chamber 110, generates an alert signal to alert the user. Specifically, the micro-controller 204 may be configured to generate the alert signal when the volume of the urine collected in the second chamber 110 reaches to the pre-determined volume of the second chamber 110.

In some embodiments, the one or more sensors 202 may include but not limited to a flow rate sensor, a capacitive sensor, and a particle sensor.

In some embodiments, the alert may be one of a vibrational alert, an audible alarm, and the like.

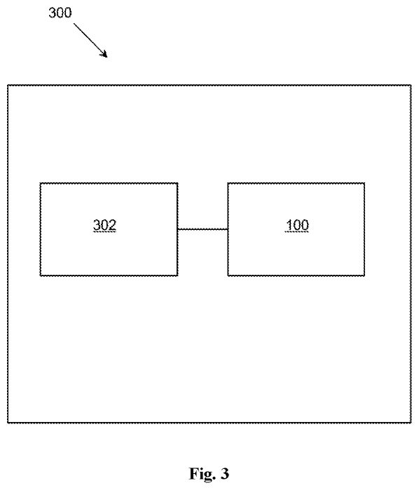

FIG. 3 illustrates a block-diagram of a system 300 to alert a user, in accordance with an embodiment herein. The system 300 may include a urostomy bag 100 and a user device 302. The user device 302 may be coupled to the urostomy bag 100 such that the micro-controller 204 generates the alert signal on the user device 302 to alert the user. The user device 302 may therefore update the filling status of the second chamber 110.

In some embodiments, the user device may include but not limited to a mobile phone, a personal digital assistant (PDA) device, a tablet, a personal-computer (PC), a screen, and other hand-held device.

In some embodiments, the user device 302 may be wirelessly coupled to the urostomy bag 100.

Thus, the urostomy bag 100 may provide following advantages that may be derived from the structural and functional aspects of the urostomy bag 100:
The urostomy bag 100 alerts the user when the volume of the second chamber 110 gets filled up to a pre-determined volume.
The urostomy bag 100 can be easily carried from one place to another.
The user device 302 is easy to carry from one place to another and thereby facilitating the user to receive the alert any location.
The urostomy bag 100 facilitates easy drainage of the urine that is accumulated within the second chamber 110 by way of the outlet valve 112.

The foregoing discussion of the present disclosure has been presented for purposes of illustration and description. It is not intended to limit the present disclosure to the form or forms disclosed herein. In the foregoing Detailed Description, for example, various features of the present disclosure are grouped together in one or more aspects, configurations, or aspects for the purpose of streamlining the disclosure. The features of the aspects, configurations, or aspects may be combined in alternate aspects, configurations, or aspects other than those discussed above. This method of disclosure is not to be interpreted as reflecting an intention the present disclosure requires more features than are expressly recited in each claim. Rather, as the following claims reflect, inventive aspects lie in less than all features of a single foregoing disclosed aspect, configuration, or aspect. Thus, the following claims are hereby incorporated into this Detailed Description, with each claim standing on its own as a separate aspect of the present disclosure.

Moreover, though the description of the present disclosure has included description of one or more aspects, configurations, or aspects and certain variations and modifications, other variations, combinations, and modifications are within the scope of the present disclosure, e.g., as may be within the skill and knowledge of those in the art, after understanding the present disclosure. It is intended to obtain rights which include alternative aspects, configurations, or aspects to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

We claim(s):

1. A urostomy bag (100) for a user, the urostomy bag (100) comprising:
a first chamber (102) disposed on a stoma of the user such that the first chamber (102) receives the urine of the user;
a tube (104) having a first end (104A) and a second end (104B) such that the first end (104A) is received within the first chamber (102) to allow passage of the urine through the tube (104);
a second chamber (110) having an inlet (110A) and an outlet (110B) and coupled to the tube (104) such that the second end (104B) is received within the inlet (110A) to allow passage of the urine from the tube (104) to the second chamber (); and
an electronic sensor unit (106) disposed between the first end (104A) and the second end (104B) of the tube (104) and configured to alert the user when volume of the urine in the second chamber (110) reaches to a pre-determined volume of the second chamber (110).

2. The urostomy bag (100) as claimed in claim 1, wherein the electronic sensor unit (106) comprising:
one or more sensors (202) configured to sense flow rate of the urine that flows through the tube (104) to measure quantity of the urine collected inside the second chamber (110);
a microcontroller (204) coupled to the one or more sensors (202) and configured to, based upon the quantity of the urine collected inside the second chamber (110), generates an alert signal to alert the user; and
a battery (206) that is coupled to the one or more sensors (202) and the microcontroller (204) and configured to supply electrical power to the one or more sensors (202) and to the microcontroller (204).

3. The urostomy bag (100) as claimed in claim 2, wherein the one or more sensors (202) comprises one of a flow rate sensor, a capacitive sensor, and a particle sensor.

4. The urostomy bag (100) as claimed in claim 2, wherein the microcontroller (204) generates the alert signal when volume of the urine collected in the second chamber (110) reaches to the pre-defined volume of the second chamber (110).

5. The urostomy bag (100) as claimed in claim 1, wherein the second chamber (110) comprising a plurality of baffles (110C) arranged within the second chamber (110) such that the plurality of baffles (110C) defines a cell inside the second chamber (110).

6. The urostomy bag (100) as claimed in claim 1, further comprising a non-return valve (108) that is disposed within the second chamber (110) and coupled to the second end (104B) of the tube (104) and adapted to prevent back-flow of the urine that passes through the tube (104).

7. The urostomy bag (100) as claimed in claim 1, further comprising:
an outlet valve (112) coupled to the outlet (110B) of the second chamber (110); and
a push button (114) coupled to the outlet valve (112), wherein upon actuation of the push button, the outlet valve (112) exhibits one of an open position and a closed position, wherein the closed position prevents drainage of the urine collected in the second chamber (110) and the open position allows drainage of the urine collected in the second chamber (110).

8. A system (300) to alert a user, the system (300) comprising:
a urostomy bag (100) comprising:
a first chamber (102) disposed on a stoma of the user such that the first chamber (102) receives the urine of the user;
a tube (104) having a first end (104A) and a second end (104B) such that the first end (104A) is received within the first chamber (102) to allow passage of the urine through the tube (104);
a second chamber (110) having an inlet (110A) and an outlet (110B) and coupled to the tube (104) such that the second end (104B) is received within the inlet (110A) to allow passage of the urine from the tube (104) to the second chamber (110); and
an electronic sensor unit (106) disposed between the first end (104A) and the second end (104B) of the tube (104) and configured to alert the user when volume of the urine in the second chamber (110) reaches to a pre-determined volume of the second chamber (110).

9. The system (300) as claimed in claim 8, wherein the electronic sensor unit (106) comprising:
one or more sensors (202) configured to sense flow rate of the urine that flows through the tube (104) to measure quantity of the urine collected inside the second chamber (110);
microcontroller (204) coupled to the one or more sensors (202) and configured to, based upon the quantity of the urine collected inside the second chamber (110), generates an alert signal to alert the user; and
a battery (206) that is coupled to the one or more sensors (202) and the microcontroller (204) and configured to supply electrical power to the one or more sensors (202) and to the microcontroller (204).

10. The system (300) as claimed in claim 9, further comprising a user device (302) coupled to the urostomy bag (100), wherein the microcontroller (204) generates the alert signal on the user device (302) to alert the user.
